(19) United States
(12) Patent Application Publication
(10) Pub. No.: US 2008/0319244 A1
(43) Pub. Date: Dec. 25, 2008

(54) Method for the Production of (E,Z)-7,8-Cyclohexadecene-1-one

(75) Inventors: Horst Surburg, Holzminden (DE); Erich Dilk, Holzminden (DE); Aurelia Reckziegel, Dormagen (DE); Walter Kuhn, Holzminden (DE)

Correspondence Address: ROYLANCE, ABRAMS, BERDO & GOODMAN, L.L.P., 1300 19TH STREET, N.W., SUITE 600, WASHINGTON,, DC, 20036, US

(73) Assignee: SYMRISE GMBH & CO. KG, Holzminden (DE)

(21) Appl. No.: 11/572,360

(22) PCT Filed: Jul. 18, 2005
(86) PCT No.: PCT/EP2005/053454

§ 371 Date: Jul. 22, 2008

(30) Foreign Application Priority Data
Jul. 21, 2004 (DE) 10 2004 035 389.1

(51) Int. Cl. C07C 5/25

(52) U.S. Cl. 585/670

(57) ABSTRACT

A process for the preparation of a (E,Z)-7,8-cyclohexadecen-1-one isomeric mixture is described, comprising the following step:
partial isomerisation of (E,Z)-8-cyclohexadecen-1-one so that the (E,Z)-7,8-cyclohexadecen-1-one isomeric mixture is formed.

The present invention relates to an improved process for the preparation of (E,Z)-7,8-cyclohexadecen-1-one.

DE 103 61 524 discloses a mixture of (E,Z)-7-cyclohexadecen-1-one and (E,Z)-8-cyclohexadecen-1-one for which a strong, clean and complex musk odour is indicated. The mixture has an elegant, uplifting and crystalline musk odour, and odour effects reminiscent of ambrette musk, for example, can be achieved therewith. The (E,Z)-7,8-cyclohexadecen-1-one isomeric mixture accordingly represents an interesting and valuable fragrance and flavouring mixture.

For the preparation of (E,Z)-7,8-cyclohexadecen-1-one. DE 103 61 524 indicates a 2-stage process starting from a mixture of 1,8- and 1,9-cyclohexadecanedione, in which first a partial reduction and then an acid dehydration are carried out. According to Example 1 of DE 103 61 524, a reaction mixture consisting of 53% (E,Z)-7,8-cyclohexadecen-1-one, 22% unreacted 1,8/1,9-cyclohexadecanedione and 22% cyclohexadecadiene is obtained. This mixture is separated by fractional distillation. While the 1,8/1,9-cyclohexadecanedione can be fed back into the process, the cyclohexadecadiene must be discarded. (E,Z)-7,8-cyclohexadecen-1-one is obtained in a distilled yield of only 35% according to this preparation process.

DE 103 61 524 additionally indicates a process for the preparation of (E,Z)-7-cyclohexadecen-1-one by olefin metathesis, in which the 1,17-octadecadien-8-one required therefor must be prepared in a complex multi-stage process.

The synthesis of a mixture of (E)-7-cyclohexadecen-1-one and (E)-8-cyclohexadecen-1-one is described in Tetrahedron, 1965, 21, 1537. Aleuritic acid (9,10,16-trihydroxypalmitic acid), protected in the form of the isopropylidene derivative, is oxidised to the dicarboxylic acid, hydrobrominated and esterified. From the resulting diester there is obtained after elimination of bromine via further stages a mixture of (E)-7-cyclohexadecen-1-one and (E)-8-cyclohexadecen-1-one.

A simple and effective process for the preparation of (E,Z)-7,8-cyclohexadecen-1-one, which in particular is suitable also for large-scale preparation, is therefore sought.

Surprisingly, it has been shown that a (E,Z)-7,8-cyclohexadecen-1-one isomeric mixture can be obtained in a simple manner, with high selectivity, by isomerisation of (E,Z)-8-cyclohexadecen-1-one. The corresponding reaction is shown schematically hereinbelow:

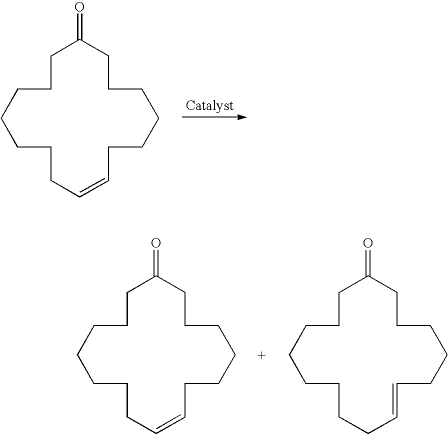

While isomerisations of aliphatic olefins, such as, for example, allyl rearrangements, are described extensively in the literature, there are no references in the literature to the displacement of double bonds in macrocyclic ring systems by only one carbon atom.

For macrocyclic alkadienes having a ring size of from 12 to 22 ring atoms, A. J. Hubert and J. Dale, in the Journal of the Chemical Society, 1963, 4091-4096, have described the isomerisation of the double bond with triethylborane at 200° C. Under these conditions, however, a double-bond isomeric mixture with random distribution of the theoretically possible double-bond isomers is obtained. In the Journal of the Chemical Society, 1965, 3118-3126, the same authors have also reported a random product distribution in the isomerisation of macrocyclic alkadienes with potassium tert.-butoxide.

In the Journal of the American Chemical Society, 1976, 98, 7102-7104, P. A. Grieco et al. describe for α-alkyl-substituted cycloalkenones having from 6 to 8 ring atoms the migration of the double bond over the ring to form the more stable α,β-unsaturated isomer by heating for 3 hours with rhodium(III) chloride trihydrate at 100° C.

It is therefore wholly surprising, and was not to be expected, that, starting from (E,Z)-8-cyclohexadecen-1-one, a selective displacement of the double bond by only one carbon atom can be achieved. In the process according to the invention, the (E,Z)-8-cyclohexadecen-1-one is preferably brought into contact with
(i) an acid or
(ii) a catalyst containing an element of sub-group VIII
   in such a manner that it isomerises partially to form the (E,Z)-7,8-cyclohexadecen-1-one isomeric mixture.

The resulting (E,Z)-7,8-cyclohexadecen-1-one isomeric mixture then contains approximately from 35 to 40% (E,Z)-7-isomer and approximately 60% (E,Z)-8-isomer. Other isomers are formed either not at all or to only a small degree. Isomerisation with random distribution of the double-bond isomers is not observed.

### (i) Acids for Use in the Process According to the Invention:

According to alternative (I) above there can be used in particular inorganic or organic protonic acids as well as acidic fixed-bed catalysts. Examples of inorganic protonic acids, which can be used individually or in a mixture, are sulfuric acid, sulfurous acid, salts of the hydrogen sulfate ion such as, for example, potassium and sodium hydrogen sulfate, sulfonic acids such as p-toluenesulfonic acid, benzenesulfonic acid, methanesulfonic acid, trifluoromethanesulfonic acid, hydrochloric acid, perchloric acid, hydrobromic acid, nitric acid, phosphoric acid, salts of dihydrogen phosphate such as potassium and sodium dihydrogen phosphate. Preferred acidic catalysts are the sulfonic acids, with trifluoromethanesulfonic acid and p-toluenesulfonic acid being particularly preferred. The amount to be used depends on the particular sulfonic acid in question. For example, when trifluoromethanesulfonic acid is used, 1 wt. % (based on the (E,Z)-8-cyclohexadecen-1-one used) is already sufficient, while from preferably 10 to 30 wt. % (based on the (E,Z)-8-cyclohexadecen-1-one used) of p-toluenesulfonic acid are employed.

Further examples of acids that can be used are the organic protonic acids, in particular the protonic acids derived from alkanes or aromatic compounds, such as formic acid, acetic acid, propionic acid, butyric acid, 2-ethylhexanoic acid, benzoic acid, oxalic acid, citric acid, tartaric acid, succinic acid, malic acid, maleic acid, fumaric acid and adipic acid.

It is also possible to use acids that are in the solid state of aggregation, acidic fixed-bed catalysis being particularly advantageous in this respect. The use of acidic cation exchangers as acidic fixed-bed catalysts is particularly advantageous. The group of the acidic cation exchangers includes in particular cation exchangers based on polymerisation synthetic resins having varying crosslinklng, a macroporous structure and active groups of different acid strength. There may be mentioned as weakly to strongly acidic ion exchangers based on synthetic resins in particular Lewatite® (Bayer) and Amberlite® (Rohm und Haas). It is further possible to use montmorillonites, such as, for example, the K-catalysts (name given by Südchemie to specially acid-treated montmorillonites). Acidic fixed-bed catalysts that can be used as an alternative are, for example, support materials (such as silica gel) loaded with mineral acids.

The isomerisation catalysed by an acid can be carried out both without a solvent and using an inert solvent such as, for example, cyclohexane, toluene or xylene, the latter variant being particularly preferred.

In order to carry out the acid-catalysed isomerisation at a satisfactory speed, a reaction temperature above 80° C., preferably above 100° C. and particularly preferably above 120° C. is preferably chosen. The reaction time is dependent on the reaction temperature and the other reaction conditions. For example, when carrying out the reaction with 20 wt. % (based on the (E,Z)-8-cyclohexadecen-1-one used) of p-toluenesulfonic acid at 115° C. and using toluene as solvent, from 30 to 40 hours are required for the isomeric equilibrium to be established, if the otherwise equivalent reaction is carried out at 140° C. in xylene as solvent, from 3 to 4 hours are already sufficient.

### (ii) Metal Catalysts (Catalysts Containing an Element of Sub-Group VIII) for Use in the Process According to the Invention:

According to alternative (II) above: catalysts containing an element of sub-group VIII can also be used as catalysts for the isomerisation. There can be used as elements of sub-group VIII in particular ruthenium, rhodium, palladium, osmium, iridium and platinum, with ruthenium, rhodium, palladium and iridium being particularly preferred. The mentioned catalysts can be used in elemental, metal form, and they are generally applied to a support. Preference is given to support materials such as activated carbon, aluminium oxide or silicon dioxide. The concentration of the catalysts on the support material is preferably from 5 to 10%.

In order to increase the activity and/or selectivity, the elements of sub-group VIII are preferably complexes with ligands. In the transition metal compounds, the elements of sub-group VIII are generally formally zero-valent or singly, doubly or triply positively charged. There can be used as counterions, for example, chloride, bromide, iodide, sulfate, nitrate, sulfonate or borate. Examples of suitable ligands are acetonitrile, benzonitrile, diethyl ether, carbon monoxide, tetrahydrofuran, hydrogen, amines, ketones, phosphanes, ethyl acetate, dimethyl sulfoxide, dimethylformamide and hexamethyl-phosphoric acid triamide.

In summary, therefore, preference is given to processes according to the invention in which the element of sub-group VIII is present
(a) in elemental form.
(b) in complexed or uncomplexed form as a salt, whereby it possesses a formally mono- to tri-valent oxidation stage,
   or
(c) in the form of a complex compound, whereby it is formally zero-valent.

The following catalysts can be mentioned as examples:

### Rhodium Catalysts:

rhodium(III) bromide hydrate, rhodium(III) chloride, rhodium(III) chloride hydrate, rhodium(III) iodide hydrate, rhodium(III) nitrate, rhodium(III) phosphate, rhodium(III) sulfate, rhodium(II) acetate dimer, rhodium(II) acetonylacetate, rhodium(I) bis-(1,5-cyclooctadiene)-tetrafluoroborate hydrate, rhodium(I) bis-(1,5-cyclooctadiene)-acetylacetonate, rhodium(I) bis-(1,5-cyclooctadiene)-chloride dimer, rhodium(I) bis-(1,5-cyclooctadiene)-trifluoromethanesulfonate dimer, rhodium(I) [1,4-bis-(diphenylphosphino)-butane]-(1c,5c-cyclooctadiene)-tetrafluoroborate, rhodium(I) [1,4-bis-(diphenylphosphino)-butane]-(2,5-norbornadiene)-tetrafluoroborate, rhodium(I) (2,5-norbornadiene)-perchlorate, rhodium(I) bis-(triphenylphosphine)-carbonyl-chloride, rhodium(II) trifluoroacetate dimer, rhodium(I) tris-(triphenylphosphine)-bromide, rhodium(I) tris-(triphenylphosphine)-chloride, rhodium(I) dicarbonyl-acetylacetonate, rhodium(I) dicarbonyl-chloride dimer,

### Ruthenium Catalysts:

ruthenium(III) bromide hydrate, ruthenium(III) chloride, ruthenium(III) chloride hydrate, ruthenium(III) iodide, ruthenium carbonyl, ruthenium(I) acetate polymer, ruthenium(III) acetonylacetate, ruthenium(II) (1,5-cyclooctadiene)-chloride polymer, ruthenium(II) tris-(triphenylphosphine)-chloride, ruthenium(II) tricarbonyl-chloride dimer, ruthenium(II) carbonyldihydrido-tris-(triphenylphosphine), ruthenium(III) 2,4-pentanedionate,

### Palladium Catalysts:

palladium(II) acetate, palladium(II) acetonylacetonate, palladium(II) bis-(acetonitrile)-chloride, palladium(II) bis-(benzonitrile)-chloride, palladium(II) [1,2-bis-(diphenylphosphino)-ethane]-chloride, palladium(II) bis-(tricyclohexyl-phosphine)-chloride, palladium(II) bis-(triphenylphosphine)-chloride, palladium(II) bis-(triphenylphosphine)-bromide, palladium(II) bromide, palladium(II) chloride, palladium(II) diammine-chloride, palladium(II) iodide, palladium(II) nitrate, palladium(II) 2,5-norbornadiene-chloride, palladium(II) sulfate, palladium(II) tetrammine-chloride, palladium(II) [1,1′-ferrocenylbis(diphenylphosphane)]-dichloride dichloromethane, palladium on activated carbon, palladium on aluminium oxide,

### Iridium Catalysts:

iridium acetate, iridium(III) acetylacetonate, iridium(I) bis-(triphenylphosphine)-carbonyl-chloride, iridium(III) bromide hydrate, iridium carbonyl, iridium(III) chloride, iridium(III) chloride hydrate, iridium(I) (1,5-cyclooctadiene)-acetylacetonate.

Particularly preferred catalysts are, for example, rhodium(I) bis-(triphenylphosphine)-carbonyl-chloride, palladium(II) bis-(benzonitrile)-chloride, ruthenium(II) tris-(triphenylphosphine)-chloride, iridium(I) bis-(triphenylphosphine)-carbonylchloride and palladium on activated carbon.

The isomerisation catalysed by such a metal catalyst is preferably carried out in the temperature range from 40 to 250° C.; at low temperatures, longer reaction times are necessary, and at higher temperatures, decomposition reactions can occur to a certain degree. A particularly preferred temperature range is from 80 to 180° C.

For the isomerisation, catalyst concentrations ≧0.01 wt. % (based on the (E,Z)-8-cyclohexadecen-1-one used) are employed, preferred concentrations being in the range from 0.02 to 3 wt. % and particularly preferred concentrations being in the range from 0.05 to 0.15 wt. %; a concentration of 0.1 wt. % is very particularly preferred.

In the case of elemental palladium optionally applied to a support, the concentration of palladium is preferably in the range from 0.01 to 0.15 wt. % and particularly preferably in the range from 0.02 to 0.08 wt. %, based on the weight of the (E,Z)-8-cyclohexadecen-1-one used.

The isomerisation catalysed by one of the mentioned catalysts can be carried out both with the use of an inert solvent such as, for example, toluene, xylene, cyclohexane, and without a solvent, the latter variant being particularly preferred.

### EXAMPLES

The starting material used in the isomerisation examples contains 98% (E,Z)-8-cyclohexadecen-1-one, wherein 67% E-isomer and 31% Z-isomer are present.

### Examples 1 to 6

### Transisomerisation with Acid Catalysts

### Example 1

Reaction conditions: catalyst: p-toluenesulfonic acid (20 wt. %); solvent: xylene; temperature: 140° C.; reaction time: 4 hours

360 g of starting material, 72 g of p-toluenesulfonic acid monohydrate and 1400 ml of xylene am heated for 4 hours at 140° C., 7.4 g of wafer, originating from the monohydrate, first being separated off in a water separator. When the reaction is complete, washing is carried out at 60° C. using 1000 g of 5% sodium hydrogen carbonate solution. In order to improve the phase separation, 300 g of sodium chloride solution are added. 1320 g of aqueous phase am separated off. The organic phase is concentrated using a rotary evaporator to leave 418 g of residue, which are distilled on a 30 cm packed column. At a pressure of 0.82 mbar, a main fraction of 288 g of product, which corresponds to a yield of 80% of theory, is obtained at a boiling temperature of 130° C. The product has the following composition (amounts in wt. %, based on the total weight of the product):

| | Cyclohexadec-8-en-1-one | Cyclohexadec-7-en-1-one |
|---|---|---|
| E-isomer | 38.4 | 26.9 |
| Z-isomer | 24.6 | 9.2 |
| Total | 63 | 36.1 |

### Example 2

Reaction conditions: catalyst: methanesulfonic acid (20 wt. %); solvent: xylene; temperature: 140° C.; reaction time: 4 hours

50 g of (E,Z)-8-cyclohexadecen-1-one, 10 g of methanesulfonic acid and 200 ml of xylene am heated for 4 hours at 140° C. Working up is carried out analogously to Example 1. After distillation, 30 g (yield: 60% of theory) of product having the following composition are obtained (amounts in wt. %, based on the total weight of the product):

| | Cyclohexadec-8-en-1-one | Cyclohexadec-7-en-1-one |
|---|---|---|
| E-isomer | 43.5 | 27.8 |
| Z-isomer | 12.0 | 8.0 |
| Total | 55.5 | 35.8 |

### Example 3

Reaction conditions: catalyst: trifluoromethanesulfonic acid (1 wt. %); solvent: xylene; temperature: 120° C.; reaction time; 13 hours

30 g of (E,Z)-8-cyclohexadecen-1-one, 0.3 g of trifluoromethanesulfonic acid and 120 ml of xylene are heated for 13 hours at 120° C. Working up is carried out analogously to Example 1. After distillation, 24.3 g (yield: 81% of theory) of product having the following composition are obtained (amounts in wt. %, based on the total weight of the product):

| | Cyclohexadec-8-en-1-one | Cyclohexadec-7-en-1-one |
|---|---|---|
| E-isomer | 44.6 | 30.8 |
| Z-isomer | 14.2 | 8.4 |
| Total | 58.8 | 39.2 |

### Example 4

Reaction conditions: catalyst: sulfuric acid (3 wt. %); no solvent; temperature; 120° C.; reaction time: 8 hours

50 g of (E,Z)-8-cyclohexadecen-1-one and 1.5 g of concentrated sulfuric acid are heated for 8 hours at 120° C., washed with sodium hydrogen carbonate solution until neutral and then distilled in vacuo. 37.5 g (yield: 75% of theory) of product having the following composition are obtained (amounts in wt. %, based on the total weight of the product):

| | Cyclohexadec-8-en-1-one | Cyclohexadec-7-en-1-one |
|---|---|---|
| E-isomer | 37.2 | 24.4 |
| Z-isomer | 12.9 | 9.4 |
| Total | 50.1 | 33.8 |

### Example 5

Reaction conditions: catalyst: montmorillonite K 10 (33 wt. %), no solvent; temperature: 120° C.; reaction time: 8 hours

30 g of (E,Z)-8-cyclohexadecen-1-one and 10 g of montmorillonite K 10 are heated for 8 hours at 120° C. and then distilled in vacuo. 21 g (yield: 70% of theory) of product having the following composition are obtained (amounts in wt. %, based on the total weight of the product):

| | Cyclohexadec-8-en-1-one | Cyclohexadec-7-en-1-one |
|---|---|---|
| E-isomer | 49.4 | 22.5 |
| Z-isomer | 17.1 | 9.2 |
| Total | 66.5 | 31.7 |

### Example 6

Reaction conditions: catalyst: Lewatit K 2641 (20 wt. %); no solvent; temperature: 120° C.; reaction time: 8 hours

40 g of (E,Z)-8-cyclohexadecen-1-one and 8 g of Lewatit K 2641 are heated for 8 hours at 120° C. and then distilled in vacuo. 22 g (yield: 55% of theory) of product having the following composition are obtained (amounts in wt. %, based on the total weight of the product):

| | Cyclohexadec-8-en-1-one | Cyclohexadec-7-en-1-one |
|---|---|---|
| E-isomer | 54.2 | 7.1 |
| Z-isomer | 30.1 | 6.3 |
| Total | 84.3 | 13.4 |

### Examples 7 to 11

### Transisomerisation with Catalysts Based on Metals of Sub-Group VIII

### Example 7

Reaction conditions: catalyst: iridium(I) bis-(triphenylphosphine)-carbonyl-chloride (1 wt. %); no solvent; temperature: 120° C.; reaction time: 8 hours

40 g of (E,Z)-8-cyclohexadecen-1-one are heated with 0.4 g of iridium(I) bis-(triphenylphosphine)-carbonyl-chloride for 8 hours at 120° C. and then distilled in a Claisen distillation apparatus. 35.9 g (yield: 90% of theory) of product having the following composition are obtained (amounts in wt. %, based on the total weight of the product):

| | Cyclohexadec-8-en-1-one | Cyclohexadec-7-en-1-one |
|---|---|---|
| E-isomer | 43.4 | 28.5 |
| Z-isomer | 19.7 | 5.5 |
| Total | 63.1 | 34.0 |

### Example 8

Reaction conditions: catalyst: palladium(II) bisbenzonitrile-chloride (1 wt. %); no solvent; temperature: 120° C.; reaction time: 8 hours

40 g of (E,Z)-8-cyclohexadecen-1-one are heated with 0.4 g of palladium(II) bisbenzonitrile-chloride for 8 hours at 120° C. and then distilled in a Claisen distillation apparatus, 36.3 g (yield: 91% of theory) of product having the following composition are obtained (amounts in wt. %, based on the total weight of the product):

| | Cyclohexadec-8-en-1-one | Cyclohexadec-7-en-1-one |
|---|---|---|
| E-isomer | 43.0 | 19.1 |
| Z-isomer | 15.8 | 6.4 |
| Total | 58.8 | 25.5 |

### Example 9

Reaction conditions: catalyst: rhodium(III) chloride hydrate (3 wt. %); no solvent; temperature: 120° C.; reaction time: 8 hours

40 g of (E,Z)-8-cyclohexadecen-1-one are heated with 1.2 g of rhodium(III) chloride hydrate for 8 hours at 120° C. and then distilled in a Claisen distillation apparatus. 38 g (yield: 95% of theory) of product having the following composition are obtained (amounts in wt. %, based on the total weight of the product):

| | Cyclohexadec-8-en-1-one | Cyclohexadec-7-en-1-one |
|---|---|---|
| E-isomer | 55.1 | 16.3 |
| Z-isomer | 20.9 | 6.0 |
| Total | 76.0 | 22.3 |

### Example 10

Reaction conditions: catalyst: ruthenium(II) tris-(triphenylphosphine)-chloride (1 wt. %); no solvent; temperature: 120° C.; reaction time: 5 hours

30 g of (E,Z)-8-cyclohexadecen-1-one are heated with 0.3 g of ruthenium(II) tris-(triphenylphosphine)-chloride for 5 hours at 120° C. and then distilled in a bulb-tube distillation apparatus. 28.5 g (yield: 95% of theory) of product having the following composition are obtained (amounts in wt. %, based on the total weight of the product):

| | Cyclohexadec-8-en-1-one | Cyclohexadec-7-en-1-one |
|---|---|---|
| E-isomer | 41.6 | 31.5 |
| Z-isomer | 17.7 | 6.2 |
| Total | 59.3 | 37.7 |

### Example 11

Reaction conditions: catalyst: ruthenium(II) tris-(triphenylphosphine)-chloride (0.1 wt. %); no solvent; temperature: 150° C.; reaction time: 1 hour

30 g of (E,Z)-8-cyclohexadecen-1-one are heated with 0.03 g of ruthenium(II) tris-(triphenylphosphine)-chloride for 1 hour at 150° C. and then distilled in a bulb-tube distillation apparatus. 29.4 g (yield: 98% of theory) of product having the following composition are obtained (amounts in wt. %, based on the total weight of the product):

| | Cyclohexadec-8-en-1-one | Cyclohexadec-7-en-1-one |
|---|---|---|
| E-isomer | 42.7 | 30.0 |
| Z-isomer | 16.4 | 8.1 |
| Total | 59.1 | 38.1 |

### Example 12

Reaction conditions: catalyst: palladium on activated carbon (2 wt. %, Pd content: 5%, water content: 60%, corresponding to a palladium concentration of 0.04 wt. %); no solvent; temperature: 170° C.; reaction time: 23 hours

300 g of (E,Z)-8-cyclohexadecen-1-one are heated with 6 g of palladium (5% on activated carbon, water content 60%) for 23 hours at 170° C. and then distilled in a Claisen distillation apparatus. 282 g (yield: 94% of theory) of product having the following composition are obtained (amounts in wt. %, based on the total weight of the product):

| | Cyclohexadec-8-en-1-one | Cyclohexadec-7-en-1-one |
|---|---|---|
| E-isomer | 44.9 | 25.0 |
| Z-isomer | 16.4 | 7.3 |
| Total | 61.3 | 32.3 |

1. A process for the preparation of a (E,Z)-7,8-cyclohexadecen-1-one isomeric mixture, comprising the following step:
effecting partial isomerisation of (E,Z)-8-cyclohexadecen-1-one so that (E,Z)-7,8-cyclohexadecen-1-one isomeric mixture is obtained.

2. A process according to claim 1, wherein the (E,Z)-8-cyclohexadecen-1-one is brought into contact with
(i) an acid or
(ii) a catalyst containing an element of sub-group VIII
in such a manner that it isomerises partially to form the (E,Z)-7,8-cyclohexadecen-1-one isomeric mixture.

3. A process according to claim 2, wherein the acid is selected from the group consisting of: inorganic protonic acids, organic protonic acids and acidic fixed-bed catalysts.

4. A process according to claim 3, wherein the inorganic protonic acid is selected from the group consisting of sulfuric acid and sulfonic acids, and the acidic fixed-bed catalyst is selected from the group consisting of the acidic canon exchangers.

5. A process according to claim 4, wherein the sulfonic acid is selected from the group consisting trifluoromethanesulfonic acid and p-toluenesulfonic acid.

6. A process according to claim 4, wherein the acidic cation exchanger is selected from the group consisting of Lewatites, Amberlites and montmorillonites.

7. A process according to claim 2, wherein the element of sub-group VIII is selected from the group consisting of ruthenium, rhodium, palladium and iridium.

8. A process according to claim 2, wherein the element of sub-group VIII is present:
(a) in elemental form,
(b) in complexed or uncomplexed form as a salt, whereby it possesses a formally mono- to tri-valent oxidation stage,
or
(c) in the form of a complex compound, whereby it is formally zero-valent.

9. A process according to claim 8, wherein the element of sub-group VIII is present in the form of a salt that is selected from the group consisting of rhodium(III) chloride hydrate, palladium(II) bis-(benzonitrile)-chloride, ruthenium(II) tris-(triphenylphosphine)-chloride, iridium(I) bis-(triphenylphosphine)-carbonyl-chloride and palladium on activated carbon.

10. A process according to claim 1, wherein the isomerisation is carried out at a temperature in the range from 40 to 250° C.
